(19) United States
(12) Patent Application Publication
(10) Pub. No.: US 2015/0202144 A1
(43) Pub. Date: Jul. 23, 2015

(54) Water Activated Shaving Wipe

(71) Applicant: Melania Bodea, Mount Vernon, MO (US)

(72) Inventor: Melania Bodea, Mount Vernon, MO (US)

(21) Appl. No.: 14/598,316

(22) Filed: Jan. 16, 2015

Related U.S. Application Data
(60) Provisional application No. 61/928,827, filed on Jan. 17, 2014.

(51) Int. Cl. A61K 8/92; A61K 8/36; A61Q 9/02; A61K 8/34; A61K 8/49; A61K 8/02; A61K 8/41

(52) U.S. Cl. CPC A61K 8/925; A61K 8/0208; A61K 8/361; A61K 8/41; A61K 8/345; A61K 8/4993; A61Q 9/02; A61K 2800/87; A61K 2800/592

(57) ABSTRACT

A solvent activated shaving wipe includes a substrate, such as a cloth having a generally porous construction. The shaving wipe includes a dehydrated shaving cream composition applied or impregnated into the substrate that is dry from the point of manufacturing, packaging, and distribution. The shaving cream composition includes stearic acid, lanolin, glycerin, polyoxyethylene, sorbitan monostearate, but specifically does not include a solvent.

### REFERENCE TO RELATED APPLICATIONS

This application claims the benefit of provisional patent application U.S. Ser. No. 61/928,827 filed Jan. 17, 2014 titled Water Activated Shaving Wipe, the provisional patent application being incorporated by reference in full herein.

### BACKGROUND OF THE INVENTION

This invention relates generally to functional wipes and, more particularly, to a wipe that is substantially dry until activated to produce a lathered shaving composition.

Over the road truck drivers face certain life challenges. They sometimes essentially “live” in their truck for multiple hours or days or even permanently. For instance, semi-trucks lack bathrooms, showers, and kitchen facilities common to traditional housing options. This requires truck drivers and passengers to make frequent stops to use appropriate facilities. The task of locating facilities that include a shower or even a sink sufficient for use in shaving can be a difficult and non-trivial task. To be sure, the presence of a shower or bathtub facility for use by a woman who desires to shave her legs is particularly challenging and often results in a woman having to shave without adequate ability to use shaving cream, to rinse with running water, and the like.

Therefore, it would be desirable to have a water activated shaving wipe that produces lathered shaving cream only when activated by water. Further, it would be desirable to have a water activated shaving wipe in which shaving cream remains in a dehydrated state until water is applied and the wipe is agitated, such as by a rubbing or squeezing contact of a user. In addition, it would be desirable to have a water activated shaving wipe that may be stored with multiple wipes in a clean and dry package.

### SUMMARY OF THE INVENTION

A water activated shaving wipe according to the present invention includes a substrate, such as a cloth having a generally porous construction. The shaving wipe includes a dehydrated shaving cream composition applied or impregnated into the substrate that is dry from the point of manufacturing, packaging, and distribution. The shaving cream composition may include stearic acid, lanolin, glycerin, polyoxyethylene, sorbitan monostearate, but specifically does not include water.

Therefore, a general object of this invention is to provide a water activated shaving wipe that includes a wipe coated or impregnated with a shaving cream composition that is prepared without the element of water.

Another object of this invention is to provide the water activated shaving wipe, as aforesaid, that is activated to produce a lathered or foamed composition when water is applied to the wipe and agitated.

Still another object of this invention is to provide the water activated shaving wipe, as aforesaid, that can be packaged with multiple wipes in a dry package for individual use.

Yet another object of this invention is to provide the water activated shaving wipe, as aforesaid, that enables a person to shave even with limited or no access to traditional sink or shower facilities available.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a perspective view of a water active shaving wipe according to a preferred embodiment of the present invention;

FIG. 2a is a top view of the shaving wipe as in FIG. 1; and

FIG. 2b is a sectional view taken along line 2b-2b of FIG. 2a.

### DESCRIPTION OF THE PREFERRED EMBODIMENT

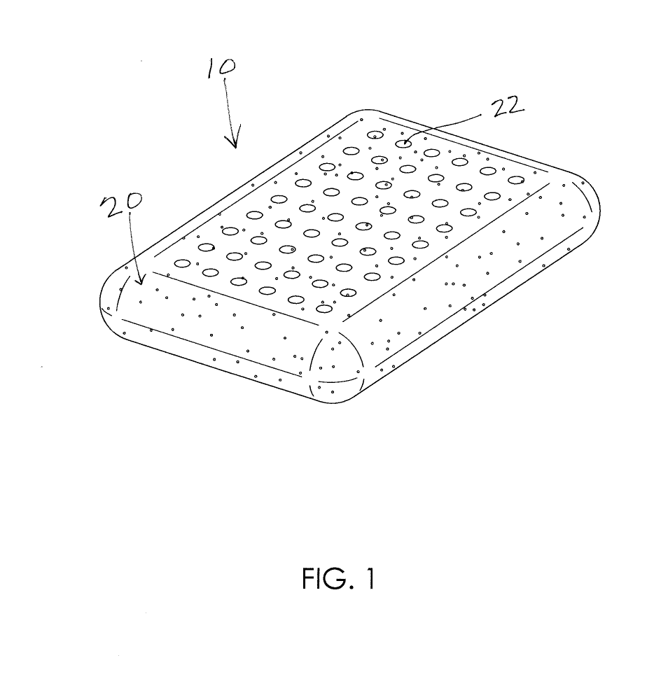

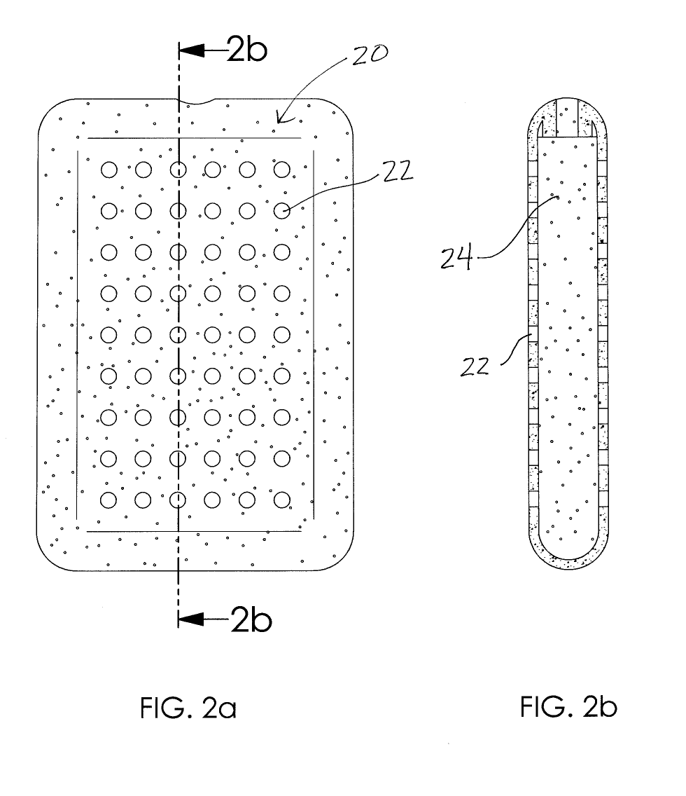

A solvent activated shaving wipe according to a preferred embodiment of the present invention will now be described with reference to FIGS. 1 to 2b of the accompanying drawings.

The water activated shaving wipe 10 includes a substrate 20 configured to be held by a user, such as a washcloth, cleaning wipe, towelette, sponge, or the like. Preferably, the substrate includes a generally porous construction capable of absorbing another substance that may applied to or coated thereon. More particularly, the substrate may include holes 22, recesses, cracks, pockets, or cavities 24 configured to receive a dehydrated composition 30, such as in the form of powder or granular materials that will be activated at a later time with a solvent, as will be described further below.

The solvent activated shaving wipe 10 includes a composition of a shaving solution in a dehydrated state and that can be selectively activated with a solvent. The shaving solution may include surfactants, humectants, conditioning agents, lubricants, and emulsifiers. For example, in one embodiment, a dehydrated composition of shaving cream may include about 5-10% by weight stearic acid, about 1-5% by weight triethanolamine, about 3-8% by weight lanolin, about 1-5% by weight glycerin, and about 3-9% by weight polyoxyethylene sorbitan monostearate.

In one aspect of the invention, the composition may include about 8% by weight stearic acid, about 4% by weight triethanolamine, about 5% by weight lanolin, about 2% by weight glycerin, and about 6% by weight polyoxyethylene sorbitan monostearate.

In another aspect of the invention, the composition may include about 8.2 percent stearic acid, about 3.7 percent triethanolamine, about 5 percent lanolin, about 2 percent glycerin, and about 6 percent polyoxyethylene sorbitan monostearate, all by weight. In a hydrated form, the formulation would include about 80% solvent, such as water. But in the dehydrated form, no solvent is included.

With specific reference to the ingredients, stearic acid is a major ingredient in the production of soap; triethanolamine has soap characteristics. Triethanolamine is used in products such as liquid laundry detergents, dishwashing liquids, general cleaners, hand cleaners, polishes, metalworking fluids, paints, shaving cream and printing inks. Both stearic acid and triehtanolamine are surfactants, or surface acting agents that reduce the surface tension between two components. Humecants are moisturizers and are often used in the cosmetic industry to prevent loss of moisture. Lanolin, a humectant, is a wax having waterproofing characteristics and is often used in skin care products. Polyoxyethylene sorbitan monostearate is an emulsifier, used in cosmetics to promote dispersion of oil in water.

Other surfactants may include, for example, ammonium, pyridinium, palmytic acid, lauric acid, and coconut oil. Other humectants may include, for example, propylene glycol, sorbitol, hexylene glycol, butylene glycol, urea, and alpha hydroxyacids. Other emulsifiers may include, for example, polysorbate 80, laureth-4, potassium cetyl sulfate, and xantham gum.

In use, one wipe or a plurality of wipes may be coated or impregnated at the point of manufacturing with the dehydrated shaving cream. The wipes may be packaged in a dry packaging unit for use at a time when shaving is desired but presumably when a traditional bathtub, sink, or shower is not available for use with conventional shaving cream. A wipe may be substantially soaked with a solvent and the wipe agitated by rubbing it with pressure against the user's leg or other body location. With the addition of the sovlent, the shaving composition will lather up and shaving may be commenced. The lathered cream may bubble up from the holes 22 and cavities 24 of the porous substrate 20.

It is understood that the dehydrated shaving wipes enable a user to apply a controlled amount of shaving cream to an area of the skin intended to be shaved. This can be done repeatedly rather than applying a large amount of shaving cream on, say, an entirety of the user's legs which may result in a substantial mess due to not being in a shower or even a restroom. For instance, this invention is better suited for shaving while in the sleeper portion of a semi-truck cab. Just apply the solvent to a single wipe and lather up a portion of a total area to be shaved.

It may be preferable for the solvent to be water, although other solvents that may be used include ethanol, acetic acid, and peroxide, for example.

It is understood that while certain forms of this invention have been illustrated and described, it is not limited thereto except insofar as such limitations are included in the following claims and allowable functional equivalents thereof.

1. A shaving wipe, comprising:
a substrate having a generally porous construction; and
a dehydrated composition deposited on the substrate, wherein a solvent applied to the substrate activates the composition, resulting in a foaming cream.

2. The wipe of claim 1, wherein the dehydrated composition comprises at least one ingredient selected from the group consisting of: surfactants, humectants, conditioning agents, lubricants, and emulsifiers.

3. The wipe of claim 1, wherein the dehydrated composition further comprises at least one surfactant selected from the group consisting of: stearic acid, theiethanolamine, pyrdinium, palmytic acid, lauric acid, and coconut oil.

4. The wipe of claim 3, wherein the solvent is water, ethanol, acetic acid, or peroxide.

5. The wipe of claim 4, wherein the dehydrated composition further comprises at least one humectant selected from the group consisting of: lanolin, glycerin, propylene glycol, sorbitol, hexylene glycol, butylene glycol, urea, and alpha hydroxy acids.

6. The wipe of claim 5, wherein the dehydrated composition further comprises at least one emulsifier selected from the group consisting of: polyoxyethylene sorbitan monostearate, polysorbate 80, laureth-4, potassium cetyl sulfate, and xantham gum.

7. The wipe of claim 6, wherein the dehydrated composition further comprises an aesthetic agent selected from the group consisting of: sandalwood, cedar, rosemary, jasmine, rose, lavender, eucalyptus, lime, peppermint, watercress, lemongrass, sage, and orange flower.

8. The wipe of claim 7, wherein the dehydrated composition comprises about 5-10% by weight stearic acid, about 1-5% by weight triethanolamine, about 3-8% by weight lanolin, about 1-5% by weight glycerin, and about 3-9% by weight polyoxyethylene sorbitan mono stearate.

9. The wipe of claim 8, wherein the dehydrated composition comprises about 8% by weight stearic acid, about 4% by weight triethanolamine, about 5% by weight lanolin, about 2% by weight glycerin, and about 6% by weight polyoxyethylene sorbitan mono stearate.

10. The wipe of claim 9, wherein the wipe is impregnated with an effective amount of the dehydrated composition.

11. The wipe of claim 10, wherein the substrate is a washcloth, a cleaning wipe, a towelette, or a sponge.

12. The wipe of claim 11, wherein the solvent is water.

13. The wipe of claim 1, wherein the dehydrated composition comprises about 5-10% by weight stearic acid, about 1-5% by weight triethanolamine, about 3-8% by weight lanolin, about 1-5% by weight glycerin, and about 3-9% by weight polyoxyethylene sorbitan mono stearate.

14. The wipe of claim 13, wherein the dehydrated composition comprises about 8% by weight stearic acid, about 4% by weight triethanolamine, about 5% by weight lanolin, about 2% by weight glycerin, and about 6% by weight polyoxyethylene sorbitan mono stearate.

15. The wipe of claim 14, wherein the substrate is a washcloth, a cleaning wipe, a towelette, or a sponge.

16. The wipe of claim 15, wherein the solvent is water.

17. A shaving cream kit, comprising a plurality of wipes packaged in a dry package for individual use, wherein each of the plurality of wipes is impregnated or coated with a dehydrated composition, the composition comprising about 8% by weight stearic acid, about 4% by weight triethanolamine, about 5% by weight lanolin, about 2% by weight glycerin, and about 6% by weight polyoxyehtylene sorbitan monostearate.

18. The shaving cream kit of claim 17, wherein the composition is activated when it comes into contact with water.

19. The shaving cream kit of claim 17, wherein agitation of the wipe produces a lathered composition.

20. The shaving cream kit of claim 17, wherein the composition further includes an aesthetic agent selected from the list comprising: sandalwood, cedar, rosemary, jasmine, rose, lavender, eucalyptus, lime, peppermint, watercress, lemongrass, sage, and orange flower.
